(19) United States
(12) Patent Application Publication
(10) Pub. No.: US 2022/0117756 A1
(43) Pub. Date: Apr. 21, 2022

(54) ROBUST IMPACTOR

(71) Applicant: Smith & Nephew Orthopaedics AG, Zug (CH)

(72) Inventors: Karin Franziska Blaser, Koniz BE (CH); Konrad Schaller, Grenchen SO (CH)

(21) Appl. No.: 17/428,345

(22) PCT Filed: Feb. 5, 2020
(86) PCT No.: PCT/EP2020/052881

§ 371 Date: Aug. 4, 2021

(30) Foreign Application Priority Data
Feb. 5, 2019 (GB) 1901590.8

(51) Int. Cl. A61F 2/46

(52) U.S. Cl. CPC A61F 2/4607; A61F 2002/4627; A61F 2002/4619

(57) ABSTRACT

A method and apparatus for urging a prosthetic femoral component into a desired position at a target location and apparatus for implanting a prosthetic stem into a terminal end region of bone are disclosed. The apparatus for urging a prosthetic femoral component into a desired target location comprises a rigid arm member (310) having a first end region that includes a locating member (370), a wedge element (342) that can be driven with an associated shaft member (340) along a drive axis, and a rotatable handle element (346) that is rotatable to turn a threaded region of the shaft member in a mating threaded region (348) carried by the rigid arm member. The rotatable handle element is rotatable to selectively drive the wedge member in a first drive direction or an opposite drive direction along the drive axis.

The present invention relates to a method and apparatus for securing a prosthetic stem at a target location and urging it into a terminal end region of bone during a surgical procedure. In particular but not exclusively, the invention relates to an impactor which provides robust, secure and reversable coupling to a hip stem such that it can be urged into a femur of a patient undergoing a hip replacement procedure with a high degree of reliability, precision and control.

Medical advancement has significantly increased human life expectancy and as a result there is a need to address bodily complications due to general wear throughout the lifetime of a patient. Joints, comprising hinge-like or socket-like moving parts, are particularly susceptible to such complications which typically require surgery to remedy. More particularly still, hip replacement surgery is often required in patients suffering from conditions such as osteoarthritis or following serious injury to the hips.

Hip replacement is a surgical procedure by which the hip joint is replaced by one or more prosthetic components. A person skilled in the art will understand that a hip joint comprises a ball-like femoral head which fits inside a socket-like acetabulum thereby permitting a human leg to exhibit a wide range of motion. During a hip replacement procedure, it is often necessary to remove the femoral head and replace it with a prosthetic ball-like element for fitting into a prosthetic socket-like element or into the natural acetabulum. This is typically achieved by first chiselling into the exposed upper end of the femur, following removal of the femoral head, along its elongate axis and reaming the resulting hole such that the femur comprises a cavity of desired shape, width, length and depth.

Following the introduction of a suitable femoral cavity, a prosthetic hip stem is urged into said cavity. Hip stems are elongate rigid devices usually constructed from a metallic or alloy material and comprise a stem body region and a stem neck region. Upon stem insertion, the stem body is urged fully or almost fully into the femur leaving only the neck region exposed. Prior to implantation of the stem, the cavity is filled with medical cement to secure the stem in place. Alternatively, the stem body is coated with hydroxyapatite, a major component of bone, allowing the femur to grow onto and over the stem body. The stem neck comprises an elongate protrusion connected to the stem body and a terminal end region of the stem neck is used for the attachment of a prosthetic ball head element constituting an artificial femoral head component of a hip joint.

Conventionally a hip stem has been urged into the above-mentioned femoral cavity using an impactor device. A conventional impactor is an elongate rod-like device comprising a handle at one terminal end and a coupling region for coupling with the stem at the other terminal end. The impactor allows for the stem body to be urged fully into the femur, minimises damage to the stem when urging into the femur, allows for the controlled positioning of the stem in the femur and allows for the stem position to be maintained until securement where necessary.

There is a need to provide better control of the coupling between a hip stem and an impactor in order to better control the implantation and positioning of the stem within the femur. There is also a need to provide a more robust system by which a hip stem can be urged into a cavity in a femur. There is a further need to provide better control when separating a stem and an impactor after use so as to minimise the disturbance and dislodgement of an implanted stem.

It is an aim of the present invention to at least partly mitigate one or more of the above-mentioned problems.

It is an aim of certain embodiments of the present invention to provide an apparatus for the implantation of a prosthetic stem, which optionally is a hip stem, into a terminal end of bone, which optionally is an upper terminus of a femur following removal of a femoral head wherein there is controllable, releasable and robust securement between the apparatus and the stem.

It is an aim of certain embodiments of the present invention to provide a method of robust securement between a stem, which optionally is a hip stem, and an impactor.

It is an aim of certain embodiments of the present invention to provide an apparatus for the robust coupling with a stem such that a medical mallet or other such surgical hammer like device can be used to provide impact forces to urge the stem into a femur without decoupling from the stem.

It is an aim of certain embodiments of the present invention to provide an apparatus that allows for controllable decoupling with an implanted stem so as to minimise disruption or dislodgement of the implanted stem.

It is an aim of certain embodiments of the present invention to provide an impactor in which a visual orientation/alignment aid can be utilised to help reduce rotation of a femur stem or anteversion in a patient.

It is an aim of certain embodiments of the present invention to provide an impactor which can be used with/coupled to a variety of femur stems of differing size and shape.

It is an aim of certain embodiments of the present invention to provide an impactor with at least one replaceable component such that component damage does not render the impactor as a whole unusable.

According to a first aspect of the present invention there is provided apparatus for urging a prosthetic femoral component into a desired position at a target location, comprising:
a rigid arm member having a first end region that includes a locating member;
a wedge element that can be driven, with an associated shaft member, along a drive axis; and
a rotatable handle element that is rotatable to turn a threaded region of the shaft member in a mating threaded region carried by the rigid arm member; whereby
the rotatable handle element is rotatable to thereby selectively drive the wedge member in a first drive direction or an opposite further drive direction along the drive axis.

Aptly the drive axis is associated with a longitudinal axis that is parallel and adjacent to a first arm axis associated with a first end region of the rigid arm member.

Aptly selective rotation of the handle element urges the wedge element towards an end of the first end region to thereby locate a narrowed portion of the wedge element between the first end region of the arm member and a neck region of the femoral component.

Aptly the narrowed portion of the wedge element is provided by two angled walls which provide an angled opening of the wedge element, the angled opening optionally being around 80°.

Aptly the narrowed portion of the wedge element can receive a variety of stems.

Aptly the rigid arm member is bent at a bent region intermediate the first end region and the remaining end region;

Aptly the first end region has a respective first arm axis that is offset from a corresponding further arm axis of the remaining end region by about around 15° to 40° and optionally by around 25°.

Aptly the first arm axis and the further arm axis are oriented at an angle of around 135° or 145°.

Aptly the mating threaded region comprises a threaded through bore in a protuberance of the arm member in the first end region.

Aptly the wedge element comprises a first sliding surface that faces a respective sliding surface provided by an outer surface of the first end region, and an abutment surface.

Aptly the abutment surface comprises a recessed channel region provided in an outer surface of the wedge element.

Aptly the recessed channel comprises a widened and a narrowed region, the narrowed region optionally being provided by a lip, and the sliding surface of the wedge element comprises a protruding element, which option has a predominantly T-shaped cross section, of a width that is narrower than the widened region and is wider than the narrowed region.

Aptly a primary axis associated with the first sliding surface is angled with respect to a primary axis associated with the channel region by about around 10° to 40° and optionally by around 30°.

Aptly the remaining end region comprises at least one elongate through hole.

Aptly a remaining end region comprises at least one predominantly circular through hole which optionally receives an elongate bolt.

Aptly the remaining end region comprises an end that includes a flared-out impact receiving head.

Aptly the flared-out impact receiving head is oriented at an angle of between 5° and 20° with respect to a vertical axis.

Aptly the remaining end region comprises a plurality of discrete rigid spaced apart elongate members that extend in a substantially parallel relationship.

Aptly a radially outermost surface of each elongate member includes wrinkles or corrugations.

Aptly the locating member is removable and/or replaceable.

According to a further aspect of the present invention there is provided a method of urging a prosthetic femoral component into a desired position at a target location, comprising the steps of:
locating a rigid arm member with respect to a femoral component via a locating member at a first end region of the arm member;
turning a handle at a first end of a shaft member supported by the arm member thereby driving the shaft member and a wedge element carried at a remaining end of the shaft member, along a respective drive axis;
as the shaft member is driven in a first drive direction, driving a narrowed portion of the wedge element between a region of the rigid arm member and a femoral neck portion of the femoral component thereby securing the arm member with respect to the femoral component; and
subsequently urging the prosthetic femoral head component into a desired position with respect to a femur.

Aptly the method further comprises locating a shaft of a prosthetic femoral component in a channel in a femur.

Aptly the method further comprises urging the prosthetic femoral component by hand or via a surgical mallet member by hitting a remaining end of the rigid arm member.

Aptly the method further comprises, subsequent to the prosthetic femoral component being duly located at a desired position, turning the handle member to drive the wedge member and shaft member away from the femoral neck; and
urging the arm member away from the femoral component.

Aptly the method further comprises hitting the remaining end by impacting a flared-out end of the arm member with a surgical mallet.

Aptly the method further comprises locating the rigid arm member at a presentation position via manipulation of a plurality of elongate handle members at an end of the rigid arm member distal to the locating member.

According to a further aspect of the present invention there is provided apparatus for implanting a prosthetic stem into a terminal end region of bone comprising: an elongate body region; a stem securement region connected to the elongate body region comprising a slidable wedge element, for imparting a first securing force on the stem, and a screw element for selecting the position of said wedge element wholly or partly along the elongate body region; and a stem coupling region comprising a protrusion element for coupling with the stem, wherein the protrusion element imparts a further securing force in response to the first securing force, thereby securing the stem to the apparatus.

Certain embodiments of the present invention provide a device able to couple/decouple to a variety of femur stems in a secure manner.

Certain embodiments of the present invention provide secure and controlled manipulation of femur stems.

Certain embodiments of the present invention provide a visual stem alignment guide during a hip replacement procedure.

Embodiments of the present invention will now be described hereinafter, by way of example only, with reference to the accompanying drawings in which:

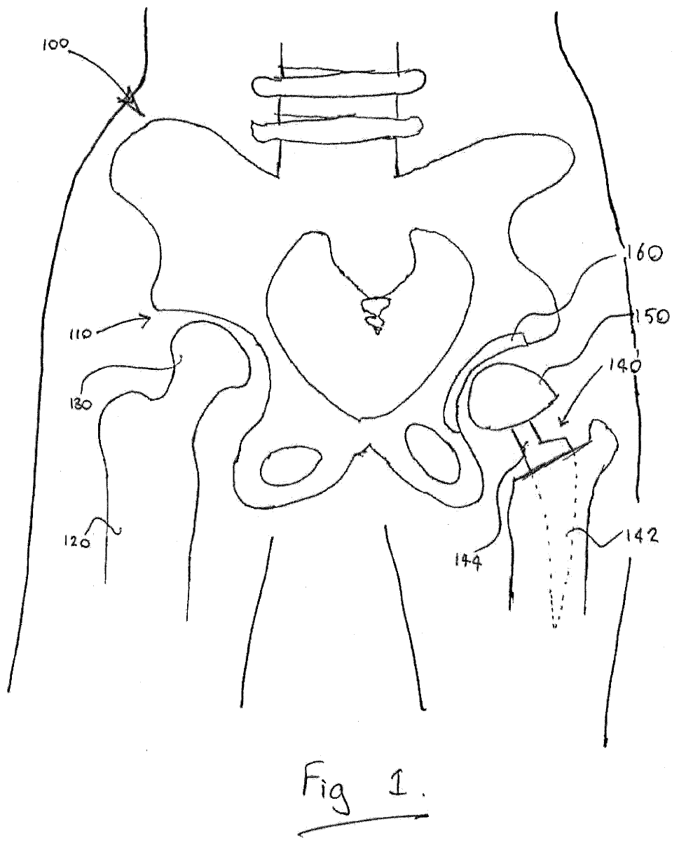

FIG. 1 illustrates a human hip and upper femur having undergone a hip replacement procedure;

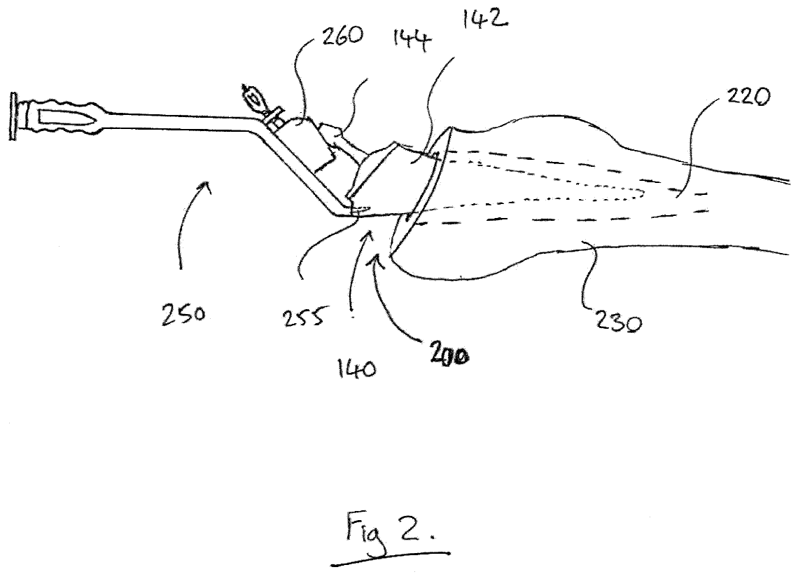

FIG. 2 illustrates hip stem implantation into a femur during the procedure;

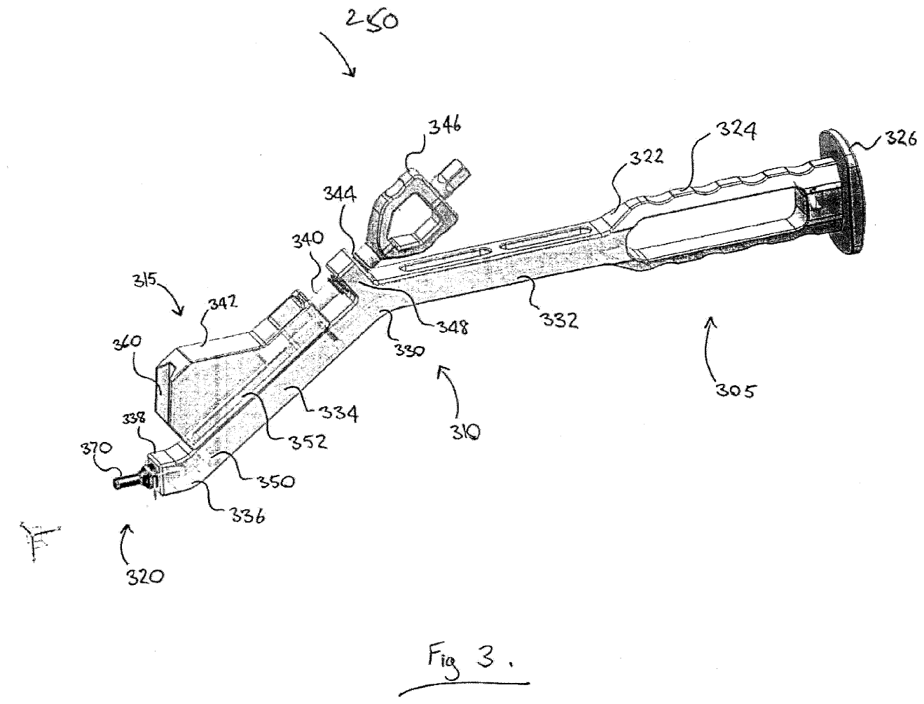

FIG. 3 illustrates a device that can be used to secure a prosthetic component at a desired location and thereafter deliver impact forces to the component;

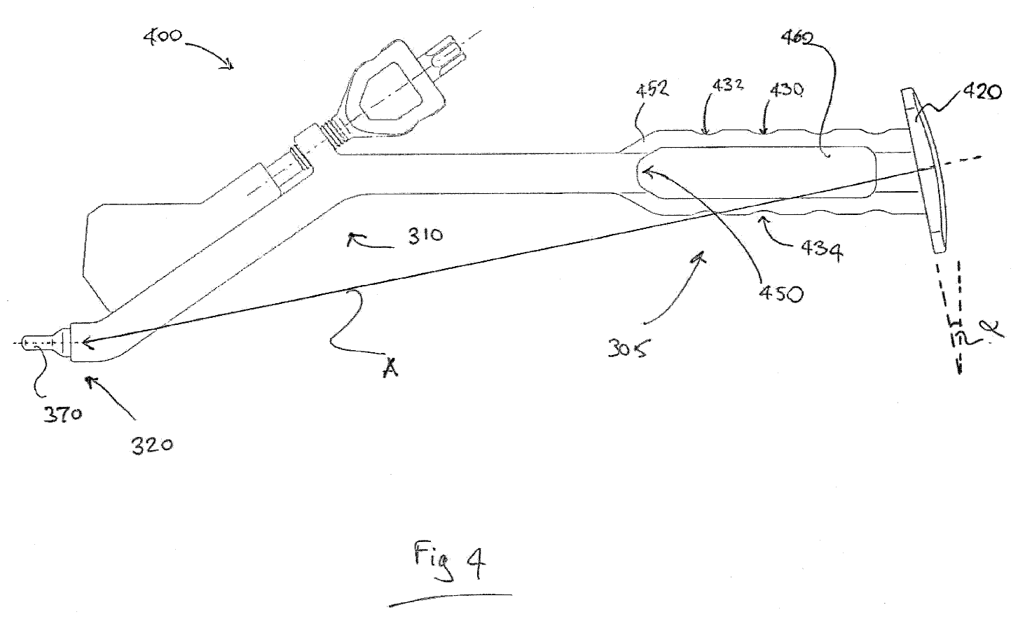

FIG. 4 illustrates a certain embodiment of a device that can be used to secure a prosthetic component at a desired location and thereafter deliver impact forces to the component comprising an angled plate element;

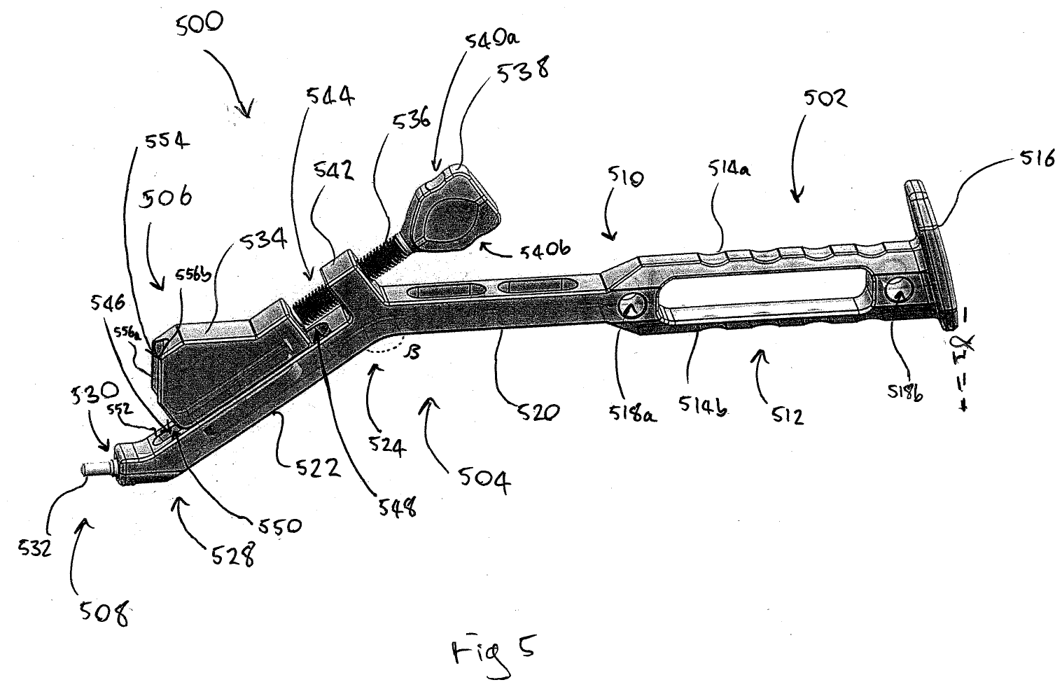

FIG. 5 illustrates an alternative impactor device;

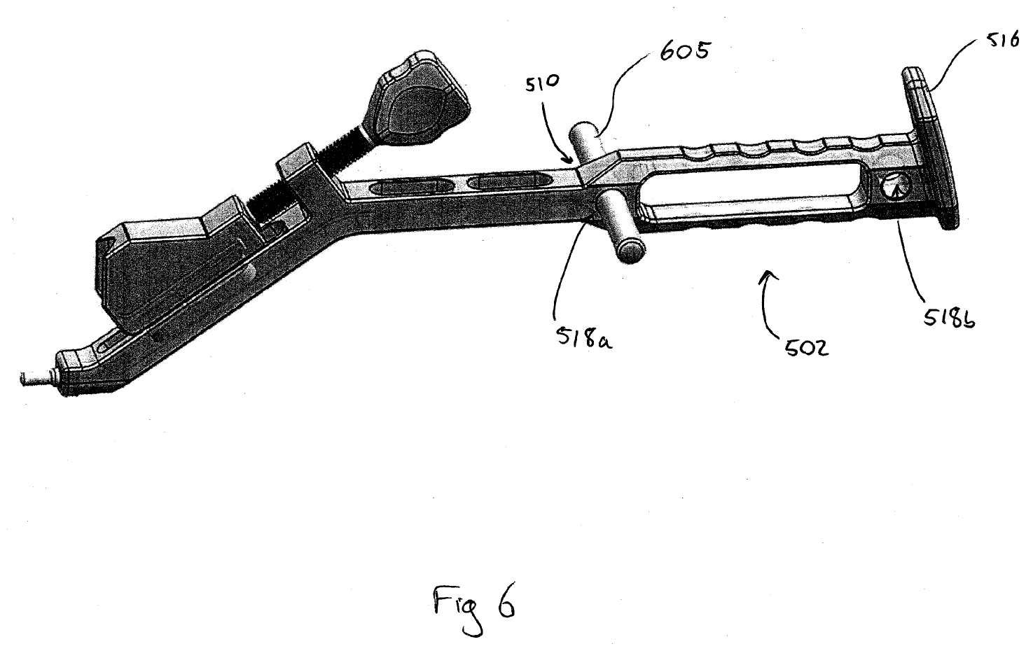

FIG. 6 illustrates the impactor device of FIG. 5, showing how a bolt can be utilised as a visual aid for aligning a stem in a surgical procedure;

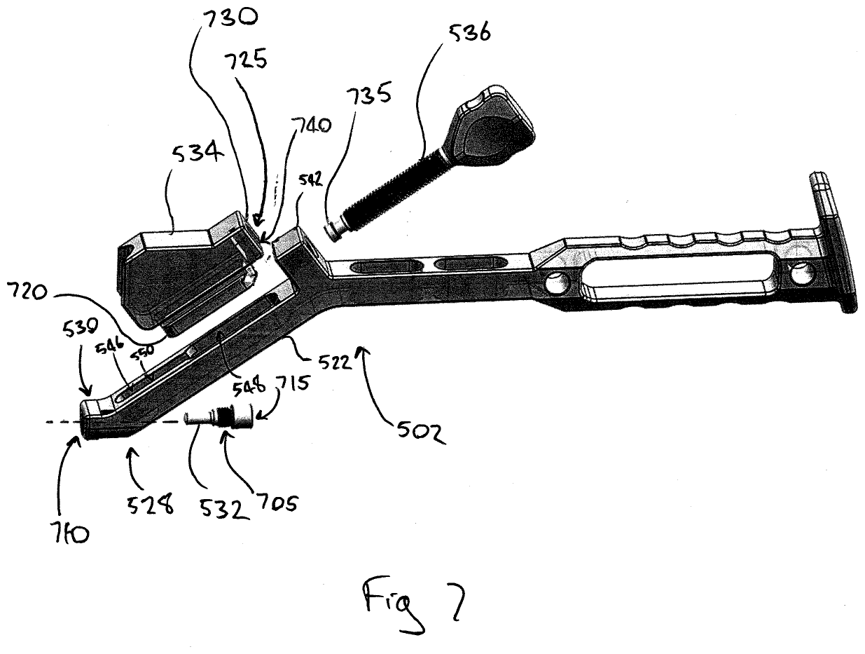

FIG. 7 illustrates the impactor device of FIG. 5, in which a protrusion element, a wedge element and a screw element are removable components; and

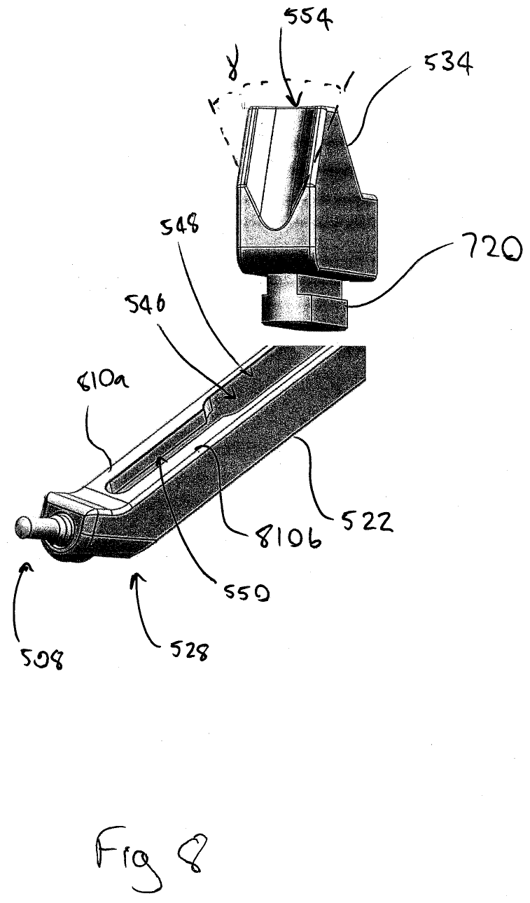

FIG. 8 illustrates the wedge element included in the impactor device of FIG. 5 and an associated recess in which the wedge element is slidably coupled to the impactor of FIG. 5 in more detail.

In the drawings like reference numerals refer to like parts.

FIG. 1 helps to illustrate the orientation of a hip stem following a hip replacement procedure. Each of the hips 100 generally includes a natural or prosthetic acetabulum 110 for the connection of the legs to the body via movement permitting joints. The natural femur 120 on the left of FIG. 1 comprises a terminal femoral head 130 which sits inside the natural acetabulum 110. During a hip replacement procedure according to certain embodiments of the present invention in which the femoral head is removed, a hip stem 140 is urged into the exposed upper femur such that the stem body 142 is embedded within the femur. The stem neck 144 is exposed and provides an attachment site for a ball head 150 which is a prosthetic replacement for the femur head. An acetabular cup 160 shown can optionally be inserted into the hip for the ball head 150 to sit inside. This is illustrated more clearly on the right-hand side of FIG. 1.

FIG. 2 helps to illustrate a stage of the stem implantation procedure using an impactor device according to certain embodiments of the present invention. At a target location 200 provided by a body part (the femur in FIG. 2) a stem 140 is urged into a cavity 220 in the femur 230 that has been produced by chiselling or reaming or by any other suitable means. The stem body 142 is urged into the cavity whilst the stem neck 144 remains exposed. During implantation, the stem 140 is coupled to an impactor 250 via a protrusion element 255 that extends into a suitable recess in the stem 140 and via a wedge element 260 that abuts against the outer surface region of the stem neck 144 most proximal to the impactor. The protrusion element is a locating member. The opposed forces of the wedge element 260 on the stem neck and the protrusion element 255 on the internal wall of the stem body 142 effectively clamp the stem 140 to the impactor 200.

FIG. 3 helps to illustrate an impactor device according to a certain embodiment of the present invention 250 for the implantation of a hip stem in more detail. The impactor contains a terminal handle region 305, an elongate body region 310, a stem securement region 315 and a terminal stem coupling region 320. The handle region 305 is connected to a terminal end of the elongate body region and comprises a tapering region 322 which tapers outwards from said elongate body region in a vertical plane resulting in a widened handle region 305 in said vertical plane. Aptly the handle region can be situated at any other suitable position within the impactor 250. Aptly the handle region forms part of the elongate body region. The handle region 305 also comprises two grip elements 324, connected between the tapering region 322 and the terminal end of the handle region 305, said grip elements 324 being separated and comprising a corrugated outer surface for gripping. Aptly any number of gripping elements 324 can be included. Aptly any outer gripping element 324 surface orientation can be utilised. Aptly the gripping elements 324 need not be separated. Aptly the handle region 305 is comprised of a metallic material. Aptly the handle region 305 is comprised of an alloy material. Aptly the handle region 305 is comprised of any other suitable material. Aptly the gripping elements 324 comprise a suitable coating for user comfort. Elongate through holes (two shown in FIG. 3) are formed in the arm member.

A terminal end of the impactor 250, can optionally comprise a plate element 326. Aptly this is comprised of a metallic material. Aptly this is comprised of an alloy material. Aptly this is comprised of any rigid polymer material. Aptly this is comprised of any other suitable material. This can optionally be a terminal end of the handle region 305. The plate element is a flared-out region. The plate element 326 can allow a user to use further medical apparatus, such as a medial mallet, to aid in urging a hip stem into a femur during a hip replacement procedure. Aptly the plate element 326 is oriented such that the elongate axis of the plate element 326 is perpendicular to the elongate axis of the handle region 305. Aptly the plate element 326 is oriented such that the direction of a first impact force, as applied to the plate element 326 in a direction perpendicular to the elongate axis of the plate element 326, results in a further force at the stem coupling region 320, said further force having a vector component analogous to the major elongate axis of the impactor 250 that is equivalent to the respective component of the first force and a vector component that is perpendicular to the major elongate axis of the impactor 250 that is opposed to the respective component of the first force.

The shape of the arm that includes the elongate body region 310 is generally characterised as being rod-like. Aptly this is comprised of a metallic material. Aptly this is comprised of an alloy material. Aptly this is comprised of any other suitable material. The elongate body region is bent and thus comprises a first curvature 330 near the centre of its length 330 such that a first significantly linear portion 332 of the elongate body region 310 is described by a first plane and is connected to the handle region 305, said first plane also describing the elongate axis of the handle region 305. A further significantly linear portion 334 of the elongate body region 310 is oriented such that it is bent away from the first plane at the first curvature 330. Aptly the arm is bent by between 10° and 80°. Aptly the radius of curvature of the first curvature 330 is 135°. Aptly any other suitable angle can be used. Aptly the impactor 250 does not contain a first bend and is straight.

The arm/elongate body region 310 also comprises a further bend/curvature 336 opposing the first curvature 330 such that an end region 338 of the elongate body region, connected to the stem coupling region 320, is described by a further plane parallel with, but spaced apart from, the first plane wherein further bend/curvature 336 is connected between the end region 338 and the further substantially linear portion 334. Aptly the further bend/curvature 336 is between 10° and 80°. Aptly the radius of curvature of the further curvature 336 is 135°. Aptly any other suitable angle can be used. Aptly the impactor 250 does not contain a further curvature 336. Aptly the impactor 250 does not contain a first curvature 330 and a further curvature 336 and the elongate body region 310 is straight.

The stem securement region 315 comprises a screw element 340 that is shaft like and a wedge element 342. Aptly the screw element comprises metallic material. Aptly the screw element comprises alloy material. Aptly the screw element comprises any other suitable material. Aptly the wedge element comprises polyether ether ketone. Aptly the wedge element comprises polymeric material. Aptly the wedge element comprises any other suitable material. The shaft/screw element 340 comprises a thread 344 and a handle element 346 at a terminal end of the screw element 340. The screw element 340 is connected to the elongate body region via a threaded flange element 348, the threaded flange element 348 has an internal bore with a threaded internal wall of thread pitch, thread depth and thread angle analogous to the thread 344 on the screw element 340, said threaded flange element protruding from the elongate body region 310 on the convex side of the further substantially linear portion 334 and in close proximity to the first curvature 330. Optionally the threaded flange element 348 can be included at any other suitable location on the elongate body region 310. Optionally the threaded flange element 348 is manufactured as part of the elongate body region 310. Optionally the threaded flange element 348 is connected to the elongate body region 310 via appropriate securement.

The screw element 340 is coupled to the elongate body element 310 via the threaded flange element 348 at the thread 344. A terminal end of the screw element is connected to the wedge element 342 such that turning the screw element 340 at the handle element 346 lengthens or shortens the separation between the wedge element 342 and the threaded flange element 348. A sliding element 350 on the underside of the wedge element 342 sits inside a sliding recess 352 in the convex side of the further substantially linear portion 334 thereby securing the wedge element 342 to the elongate body region 310 whilst also permitting a slidable ability of the wedge element 342 in response to the turning of the screw element 340 via the handle element 346. Aptly the sliding recess 352 can be at any other suitable position on the elongate body region 310. Turning the handle thus moves the shaft and the wedge member carried with the shaft selectively backwards or forwards along a longitudinal axis.

The wedge element 342 contains a slotted region 360 in an outermost surface facing the stem coupling region 320. This forms an abutment surface onto which the stem neck can intrude. The wedge element 342 thus imparts a first securing force on the stem neck which can be increased by turning the screw element 340 such that the separation between the wedge element 342 and the threaded flange element 348 is increased. The walls on either side of the slotted region 360 prevent the rotation or misalignment of the stem both when securing the stem to the impactor 250 and during implantation of the stem into a femur thereby improving the precision of the hip replacement procedure.

The stem coupling region 320 comprises a protrusion element 370 which intrudes into a recess in a stem. The protruding peg thus behaves as a locating member. Aptly the recess is included in the stem by design. Aptly the recess in the stem is created or modified by the user to be compatible with the protrusion element 370. Aptly the protrusion element is removable and can be replaced with further protrusion elements to suit a variety of stems. The protrusion element 370 imparts a further securing force on the inner wall of the stem recess in response to, and substantially, partly or wholly opposing, the first securement force. Therefore, as the first securing force is increased by turning the screw element 340, the further securing force also increases. The combination of these forces thereby secures the stem to the impactor 300.

The combination of the stem securement region 315 and the stem coupling region 320 then provides a more robust method of stem securement to the impactor 250 than is currently available via conventional techniques. This allows for a more precise and more robust method of stem insertion into a femur. This securement allows for the use of medical mallets to urge the stem into the femur via the impactor 250 whereby the energy provided by the mallet is not sufficient to overcome the first and further securement forces. The present invention also provides a more controlled method of impactor removal from the stem post implantation as opposed to conventional methods. In certain embodiments of the present invention a user can simply turn the screw element 340 such to reduce the first and further securing forces thereby releasing the implanted stem with minimal risk of disturbing or dislodging it or the prosthetic femoral component.

FIG. 4 illustrates a certain embodiment of the present invention 400, different in certain respects to that shown in FIG. 3, in which an angled plate element 420 is connected at an end of the handle region 305 at an inclination angle α such that a first impact force incident perpendicular to the elongate axis of the angled plate element 420 and generally at the centre of the angled plate element 420 results in a further impact force through the protrusion element 370 in the stem coupling region 320 with a direction substantially along the elongate axis of the protrusion element. The direction of the first impact force and the position of the further impact force at indicted by an arrow A. Aptly the inclination angle α is between 5° and 20°. Aptly the inclination angle α is 11°. Aptly the inclination angle α is any other suitable angle. Aptly the first impact force is provided by hand. Aptly the first impact force is provided by a medical mallet or similar device. Aptly the first impact force is provided by any other suitable method.

The handle region 305 comprises grip elements 324 comprising a corrugated outer gripping surface. Aptly the distance between a first innermost point of the gripping surface 430 and a further innermost point of the gripping surface 432 is between 10 mm and 30 mm. Aptly the distance between a first innermost point of the gripping surface 430 and a further innermost point of the gripping surface 432 is 20 mm. Aptly any other suitable distance between a first innermost point of the gripping surface 430 and a further innermost point of the gripping surface 432 can be utilised. Aptly the distance between the first innermost point of the gripping surface 430 and an opposed innermost surface of the gripping surface 434 is between 10 mm and 40 mm. Aptly the distance between the first innermost point of the gripping surface 430 and an opposed innermost surface of the gripping surface 434 is 27 mm. Aptly any other suitable distance between the first innermost point of the gripping surface 430 and an opposed innermost surface of the gripping surface 434 can be used. Aptly The tapering region 322 is located between grip elements 324. Aptly the tapering angle 450, located between tapering elements 452, is between 40° and 80°. Aptly the tapering angle 450 is 60°. Aptly any other suitable tapering angle 450 can be used. Aptly the tapering elements 452 comprise a thickness between 5 mm and 15 mm. Aptly the tapering elements 452 comprise a thickness of 9 mm. Aptly the tapering elements 452 comprise any other suitable thickness. Aptly there is a handle recess 460 between gripping elements. Aptly the width of the handle recess 460, whereby the width is the axis perpendicular to the elongate axis of the handle region 305, is between 10 mm and 30 mm. Aptly the width of the handle recess 460 is 18 mm. Aptly the width of the handle recess is of any other suitable length. Aptly the handle region 305 comprises a length between 50 mm and 200 mm. Aptly the handle region comprises a length of 110 mm. Aptly the handle region is any other suitable length.

Aptly the elongate body region 310 is of length between 200 mm and 400 mm. Aptly the elongate body region 310 has a of length of 305 mm. Aptly the elongate body region has any other suitable length. Aptly the elongate body region comprises a further linear portion 334 of length between 50 mm and 200 mm. Aptly the elongate body region comprises a further linear portion 334 of length 120.8 mm. Aptly the elongate body region comprises a further linear portion 334 of any other suitable length.

FIG. 5 illustrates a still further impactor device 500 according to an alternative embodiment of the present invention. The impactor illustrated in FIG. 5 is in some ways similar to the impactors illustrated in FIGS. 3 and 4 and includes a terminal handle region 502, an elongate body region 504, a stem securement region 506 and a stem coupling region 508. The handle region 502 is connected to a terminal end of the elongate body region 504 and comprises a tapering region 510 which tapers outwards from said elongate body region 504 in a vertical plane resulting in a widened handle region 512 in said vertical plane. Aptly the handle region can be situated at any other suitable position within the impactor 500. Aptly the handle region 502 forms part of the elongate body region. The handle region 502 also comprises two grip elements 514a, 514b, connected between the tapering region 510 and a terminal end of the handle region 502, said grip elements being separated and comprising a corrugated outer surface for gripping. Aptly any number of gripping elements can be included. Aptly any outer gripping element surface orientation can be utilised. Aptly the gripping elements need not be separated. Aptly the handle region 502 is comprised of a metallic material. Aptly the handle region is comprised of an alloy material. Aptly the handle region is comprised of a polymeric material. Aptly the handle region is comprised of any other suitable material. Aptly the gripping elements comprise a suitable coating for user comfort. Elongate through holes (two shown in FIG. 5) are formed in the arm member and may help reduce the impactor weight.

With similarity to the impactor illustrated in FIG. 4, a terminal end of the terminal handle region 502 comprises a plate element 516 located at a terminal end of the impactor. Aptly this is comprised of a metallic material. Aptly this is comprised of an alloy material. Aptly this is comprised of any rigid polymer material. Aptly this is comprised of any other suitable material. The plate element provides a flared-out region. The plate element 516 can allow a user to use further medical apparatus, such as a medial mallet, to aid in urging a hip stem into a femur during a hip replacement procedure. It can be seen in FIG. 5 that the flared-out region provided by the plate element 516 extends substantially greater in a vertically upward direction from the terminal handle than in a vertically downward direction or in a horizontal direction. This arrangement of the plate element 516 may restrict the angles at which a medial practitioner, surgeon or any other user of the impactor can impart forces to/on the impactor via the plate element 516 and may limit any subsequent unwanted torque resulting from imparting forces on a wider flared-out region (at a relatively greater horizontal distance from the central axis of the impactor) for example. Limiting the horizontal and vertically downward directions of the flared-out region provided by the plate element 516 may also help reduce misuse of the impactor 500, for example hitting the rear of the plate (either by hand or using equipment such as a mallet) to remove the impactor from a coupled stem. Such actions may otherwise damage the impactor and/or stem and may provide unwanted and possibly damaging forces to a patient's femur or other bodily components.

With similarity to the impactor illustrated in FIG. 4, the plate element 516 is oriented at an angle that is not perpendicular with respect to the major axis defined by the handle region 502 of the impactor. A vertical (non-inclined) knock plate 516 could of course be used along with one or more of the remaining features shown in FIG. 5 impactor 500. The plate element shown in FIG. 5 is oriented such that the an angle made by the major axis of the terminal handle region 502 and the major axis of the plate element 516 is acute in an anticlockwise direction and is obtuse in a clockwise direction (as depicted in FIG. 5). Aptly the angle of orientation of the plate element 516 is between 10° and 20° with respect to a vertical axis (perpendicular with the major axis of the impactor handle region 502) or between 70° and 80° with the respect to the major axis of the impactor handle region 502. Aptly the inclination angle α is between 5° and 20°. Aptly the inclination angle is 11°. Aptly the angle of orientation of the plate element 516 is about around 13° (inclination angle) with respect to a vertical axis (perpendicular with the major axis of the impactor handle region 502) or about around 77° with the respect to the major axis of the impactor handle region 502. Aptly the angle of orientation of the plate element 516 is about around 14.3° (inclination angle) with respect to a vertical axis (perpendicular with the major axis of the impactor terminal handle region 502) or about around 76.7° with the respect to the major axis of the impactor terminal handle region 502. With similarity to the impactor illustrated in FIG. 4, the inclination of the plate element 516 allows for the suitable transmission of a force incident on the plate element to a coupled stem.

The terminal handle region 502 additionally includes two circular through-holes 518a, 518b. One circular through-hole is located proximate to the plate element 518b and one circular through-hole is located proximate to/within the tapering region 518a. Aptly a circular through-hole is provided at any other suitable position within the impactor. Aptly only a single circular through-hole is utilised. Aptly any number of through-holes can be utilised. Aptly these through-holes can be of any suitable shape and size. Elongate bolts (not shown in FIG. 5) can be inserted into one or both of these circular through-holes 518a, 518b such that the bolt extends out of both sides of the impactor 500. The bolts can thus be used as a visual guide/aid during implantation of a hip stem. This may help orient a stem in a target femur with more precision and may help reduce aberrant rotation of the stem thereby helping to limit/prevent anteversion in patients undergoing such a surgical procedure. Aptly the bolts are comprised of a metallic material. Aptly the bolts are comprised of an alloy material. Aptly the bolts are comprised of a polymeric material. Aptly the bolts are comprised of any other suitable material. Aptly the bolts are coated in high visibility paint or any other suitable coating. Aptly any other suitable equipment can be attached to the impactor device via the circular through-holes 518a, 518b. Aptly a bolt can be provided which is connected to auxiliary equipment with can be coupled to the impactor device 500. Aptly the auxiliary equipment such as a light/laser guidance/levelling system can be attached to the impactor device via the circular through-holes 518a, 518b.

The elongate body region 504 or arm region is generally rod-like. Aptly the elongate body region 504 is comprised of a metallic material. Aptly the elongate body region 504 is comprised of an alloy material. Aptly the elongate body region 504 is comprised of a polymeric material. Aptly the elongate body region 504 is comprised of any other suitable material. As is illustrated in FIG. 5, the elongate body region 504 comprises two linear portions, a first substantially linear portion 520 and a further significantly linear portion 522, which are bent with respect to each other, the first linear portion 520 being oriented axially in substantially the same plane as the terminal handle region 502 and being connected to the terminal handle region 502 via/at the tapered region 510. The elongate body potion 504 thus includes a first curvature 524 adjoining the first substantially linear portion 520 and the further substantially linear portion 522. The first curvature 524 is located around the centre of the elongate body region 504. The first substantially linear portion and the further substantially linear portion are oriented at a particular angle β which may be a radius of curvature of the first curvature. Aptly the first curvature is such that the first substantially linear portion 520 and the further substantially linear portion 522 are oriented at an angle between 100° and 200°. Aptly the first curvature 524 is such that the first substantially linear portion 520 and the further substantially linear portion 522 are oriented at an angle of around 145°. Aptly the first curvature 524 is such that the first substantially linear portion 520 and the further substantially linear portion 522 are oriented at an angle of around 135°. Aptly the first curvature 524 is such that the first substantially linear portion 520 and the further substantially linear portion 522 are oriented at any other suitable angle.

The elongate body region 504 extends from the terminal handle region 502 to the stem coupling region 506 and comprises a further curvature such 528 that an extending terminal end 530 that is an end region of the elongate body region 504 most proximate to the stem coupling region 508 (distal the terminal handle region 502), the extending terminal end 530 also being connected to the further substantially linear portion 522, is oriented to be parallel with, but in a different plane to, the first substantially linear portion 520. The further curvature 528 thus opposes the first curvature 524. Aptly the further curvature 528 is better characterised as a sharp bend. Aptly the further curvature is between 10° and 80°.

The stem coupling region 508 comprises a protrusion element/pin 532 that is a locating member which protrudes out from the extending terminal end 530 of the elongate body region along the same axis as this extending terminal end 530 of the elongate body region 504. The protrusion element 532 is thus oriented along an axis that is parallel to the major axis defined by the terminal handle region 502 and the first substantially linear portion 520 of the elongate body region 504. Aptly the protrusion element 532 comprises a metallic material. Aptly the protrusion element 532 comprises an alloy material. Aptly the protrusion element 532 comprises a polymeric material. Aptly the protrusion element 532 comprises any other suitable material. Aptly the protrusion element 532, and therefore the stem coupling region 508, is removable from the impactor. Aptly the protrusion element 532 comprises a threaded region (not shown in FIG. 5) which is mateable with a threaded bore (also not shown in FIG. 5) located through the extended terminal end 530 of the elongate body region 504, proximate to the further curvature 528.

With similarity to the impactors illustrated in FIGS. 3 and 4, the stem securement region 506 of the impactor 500 illustrated in FIG. 5 comprises a wedge element 534 and a shaft-like screw element 536. Aptly the screw element 536 comprises metallic material. Aptly the screw element 536 comprises alloy material. Aptly the screw element 536 comprises polymeric material. Aptly the screw element 536 comprises any other suitable material. Aptly the wedge element 534 comprises polyether ether ketone. Aptly the wedge element 534 comprises polymeric material. The screw element 536 comprises a screw handle 538 at a terminal end. As is illustrated in FIG. 5, the screw handle 538 is a widened/flattened region of the screw element 536 and provides a gripping portion which can be rotated by a user. The screw handle comprises a notch 540a, 540b on either side of parallel flattened edges to aid in gripping. Aptly the gripping screw handle 538 does not comprise notches on each edge. Aptly the gripping screw handle 538 comprises any number of notches on each edge. The gripping screw handle 538 shown in FIG. 5 is a solid body and does not comprise any through holes in order to help to limit a stem securing force a user can exert in operation (through rotation of the screw element 536 when the impactor 500 is coupled to a stem in a similar manner as described previously). This may help limit damage of the impactor 500, the pin/protrusion element 532 and/or a stem in use through overtightening. Aptly, if tighter securement between a stem and the impactor 500 is desired, the screw handle 538 may comprise one or more through holes in order to permit further tightening of the screw element by a user in use (such an screw handle is illustrated in FIGS. 3 and 4). A user may then be able to place an object (or fingers) through such a hole to lever tighter securement.

The screw element 538 is connectable to the elongate body portion/region 504 via a flange element 542 located on a surface of the further substantially linear portion 522 of the elongate body region 504, proximate to the first curvature 524 and on the side of the elongate body region 504 that includes the convex side of the first curvature 524. The flange element 542 comprises a threaded bore which is mateable with a threaded portion 544 of the screw element 536. Aptly the screw element 536 is removable from the flange element 542. Aptly the flange element 542 and/or the screw element 536 is provided with a catch which prevents removal of the screw element 536 from the flange element 542 (and thus from the impactor device 500). Aptly the thread pitch, depth and angle of the threaded bore in the flange element 542 and the threaded potion 544 of the screw element 536 are substantially the same. Turning the screw element 536 in a particular direction thus drives the screw element 536 towards an end of the further substantially linear portion 522 of the elongate body region 504.

The stem securement region 506 also includes a wedge element 536 that is slidably coupled to the further substantially linear portion 522 of the elongate body region 504 and is also connected to the screw element 536. Thus, by rotating the screw element 536, the wedge element 534 can be driven up or down the further substantially linear portion 522 of the elongate body region 504 along a longitudinal drive axis that is defined by the further substantially linear portion 522. The wedge element 534 is coupled to the further substantially linear portion 522 via a sliding recess 546 in the further substantially linear portion 522, the recess 546 being located on the same face of the substantially linear portion 522 onto which the flange element 542 is located. A protruding portion of the wedge element that is a sliding element extends into the recess. Sliding of the wedge element 534 is permitted by motion of the protruding portion/sliding element of the wedge element 534 in the recess 546 of the further substantially linear portion 522 of the elongate body region 504 along a longitudinal drive axis that is defined by the major axis of the further substantially linear portion 522 of the elongate body region 504. The recess 546 comprises a widened region 548 most proximate to the first curvature 534 and a narrowed region 550 most proximate to the further curvature 528. Aptly the narrowed region 550 of the recess 546 is formed from at least one overhanging lip 552 at the opening of the recess 546. The protruding portion/sliding element of the wedge element 534 has a T-shaped cross section that is narrower in width that the widened region 548 of the of the recess 546 but is wider in width than the narrowed region 550 of the recess 546. Thus, the wedge element 534 may be placed into, and removed from, the recess 546 at the widened region 548 of the recess. When the wedge element 534 is driven (by the screw element 536) to be located in the narrowed region 550 of the recess 546 (and towards the stem for secure coupling) however, the wedge element 534 is captured in the recess 546 due to the abutment of the flared out wings of the T-shaped protruding portion of the wedge element 534 against the overhanging lips 552 of the recess 546. The wedge element 534 is thus prevented from tilting away from the elongate body portion 504 in use which helps limit misalignment of a stem during a surgical procedure.

The wedge element 534 contains a slotted region 554 in an outermost surface facing the stem coupling region 508. This forms at least one abutment surface onto which a stem neck can intrude. Aptly the abutment surface or surfaces is/are provided by walls 556a, 556b of the slotted region 554. The walls on either side of the slotted region 554 prevent the rotation or misalignment of the stem both when securing the stem to the impactor 500 and during implantation of the stem into a femur thereby improving the precision of the hip replacement procedure. The walls 556a, 556b of the slotted region 554 are substantially slanted/angled and thereby provide an angled opening of the slotted region 554. The opening of the slotted region defines an opening angle which is suitable for engagement for a particular stem or a variety of stems. Aptly this angle is between 45° and 90°. Aptly this opening angle is around 80°.

The angled opening of the slotted region 554 allows the wedge element 534 to receive multiple types of stem. The wedge element 534 therefore does not have to be manufactured to be specific to a particular stem type. This is due to the narrowing of the slotted region 554 provided by the angled walls 556a, 556b and also due to the angle at which the slotted region 554 is oriented with respect to the drive axis/the axis of the further substantially linear portion 522. Instead of fitting/gripping a particular stem, the wedge element 534 bites opposing circumferential edges of a stem neck region. Aptly the biting region engages with substantially wider terminal circumferential edges of a stem neck region. Stems with differing centrum-collum-diaphyseal (CCD) angles can thus be secured in the slotted region 554 of the wedge element 534. Compatible POLARSTEM products include Valgus (CCD angle=145°), Standard (CCD angle=135°) and Lateral (CCD angle=126°) femur stems. It will be understood that stems with a lower CCD angle will be secured at a substantially upper position in the slotted region as opposed to stems with a higher CCD angle which will be secured at a substantially lower position in the slotted region 534.

FIG. 6 illustrates how a bolt can be utilised as a visual alignment aid or guide for the impactor illustrated in FIG. 5. In FIG. 6, a bolt 605 is provided through the circular through-hole 518a proximate to/in the tapering region 510 of the handle region 502. The bolt 605 substantially extends through both sides of the impactor and provides a visual levelling aid for an operator of the impactor to orient the stem by aligning each side of the bolt 605 with an arbitrary reference. Aptly, there may be provided a visual reference or marker, either on the patient or in a surgical/operation room, with which the bolt can be aligned. Aptly, an operator can manipulate the orientation of the coupled stem and impactor by handling the bolt directly. Aptly a bolt 605 can be provided through the circular through-hole 518b proximate to the plate element 516. Aptly a bolt 605 can be provided through both circular through holes 518a, 518b. Aptly a bolt can be provided through any other through-hole. Alternatively a pin like rod can be inserted in blind hole to provide a similar visual aid for helping avoid undesired rotation of the device in use.

FIG. 7 illustrates the impactor illustrated in FIG. 5 in which the wedge element 534, the screw element 536 and the protrusion element 532 are removable. It can be seen in FIG. 6 that the protrusion element 532 comprises a threaded region 705. This threaded region is mateable with a threaded region of substantially similar thread pitch, angle and depth located within a through bore 710 extending through the extended terminal end 530 of the elongate body region 502 and at least partly through the further curved region 528 to a proximate terminus of the elongate body region 502. The location and axis of the through bore is indicated with the dotted line. Aptly the protrusion element 532 can be secured within the elongate body region using an Allen wrench that is optionally a 3.5 mm Allen wrench via an interface suitable for an Allen key (which optionally is a hexagonal recess) located on the rear 715 of the protrusion element 532. A removable and securable protrusion element 534 allows for replacement of the component if damaged without a need to replace the impactor as a whole.

It can be seen in FIG. 7 that the wedge element 534 can be removed from, or coupled to, the elongate body region 502 via the widened portion 548 of the recess 546 in the further substantially linear portion 522 of the elongate body region 502 via the protruding portion 720 of the wedge element 534. FIG. 7 also illustrates a screw coupling region 725 in the wedge element 534 which is predominantly arch-shaped and includes at least one lip/jacket 730. A terminal end of the screw element comprises a flared-out portion/region 735. The screw element 536 and the wedge element can be coupled by arranging the flared-out portion 735 of the screw element 536 to sit within/behind the lip/jacket 730 of the screw coupling region 725 of the wedge element 534. The screw coupling region 725 is accessible via a channel 740 located on the under-side of the wedge element 534 which extends into/behind the jacket/lip 730. Coupling of the screw element 536 and the wedge element 534 can thus be achieved by sliding the flared-out portion 735 under the screw coupling region 725 and into the channel 740 such the flared-out 735 portion sits behind the lip/jacket 730. When the wedge element 534 and the screw element 536 are coupled to the elongate body portion 502 via the recess 546 and the flange element 542 respectively, and when the protruding portion 720 of the wedge element 534 is located within the narrowed region 550 of the recess 546, the flared-out terminal end 735 of the screw element 536 is irremovable from the screw securement region 725 of the wedge element 534 until such a time as the wedge element 534 is moved/driven to reside in the widened potion 548 of the recess 546 (by rotation of the screw element 536) at which point the wedge element 534 can be decoupled from the elongate body region 502.

FIG. 8 illustrates the wedge element 534 and the sliding recess 546 of the further substantially linear portion 522 of the impactor illustrated in FIG. 5 in more detail. FIG. 8 illustrates how the recess 546 in the further substantially linear portion includes a widened portion 548 and a narrowed portion 550, the narrowed portion 550 being most proximate to the further curvature 528 and the stem coupling region 508. As indicated, the narrowed portion 550 is provided by two opposed lips 810a, 810b included in the further substantially linear portion 522. The T-shaped cross section of the protruding portion 720 is clearly illustrated, and it will be understood that this cross section provides securement of the wedge element 534 in the recess 546 when the wedge element 534 is located within the narrowed region 550 of the recess 546. This securement prevents the wedge element 534 from being pulled away from the further substantially linear portion 522 of the elongate body portion/region in use. Aptly the protruding portion 720 of the wedge element comprises an L-shaped cross section. Aptly the protruding portion 720 of the wedge element comprises any other suitable cross section.

FIG. 8 also helps illustrate the slotted region 554 of the wedge element. The angled opening of the slotted region provided by the angled orientation of the walls is clearly illustrated, the opening angle being indicated by y. It will be understood how a circumferential edge of a portion of stem neck can be gripped by the angled walls of the slotted region 554. The stem is thus gripped at two points on the circumferential edge of the neck region. It will be understood from FIG. 8 how stems with varying CCD angles can be gripped at varying position along the slotted region 554 provided the neck region of a stem includes a circumferential edge that is wider than the narrowest width of the slotted region 554. Aptly the opening angle of the slotted region 554 is between 45° and 90°. Aptly this opening angle is around 80°.

Throughout the description and claims of this specification, the words “comprise” and “contain” and variations of them mean “including but not limited to” and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of the features and/or steps are mutually exclusive. The invention is not restricted to any details of any foregoing embodiments. The invention extends to any novel one, or novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

1. An apparatus for urging a prosthetic femoral component into a desired position at a target location, comprising:
a rigid arm member having a first end region that includes a locating member;
a wedge element that can be driven, with an associated shaft member, along a drive axis; and
a rotatable handle element that is rotatable to turn a threaded region of the shaft member in a mating threaded region carried by the rigid arm member; whereby
the rotatable handle element is rotatable to thereby selectively drive the wedge member in a first drive direction or an opposite drive direction along the drive axis.

2. The apparatus as claimed in claim 1, wherein the drive axis is associated with a longitudinal axis that is parallel and adjacent to a first arm axis associated with a first end region of the rigid arm member.

3. The apparatus as claimed in claim 1, wherein selective rotation of the rotatable handle element urges the wedge element towards an end of the first end region to thereby locate a narrowed portion of the wedge element between the first end region of the arm member and a neck region of the femoral component.

4. The apparatus as claimed in claim 3 wherein, the narrowed portion of the wedge element is provided by two angled walls which provide an angled opening of the wedge element.

5. The apparatus as claimed in claim 3, wherein the narrowed portion of the wedge element can receive a variety of stems.

6. The apparatus as claimed in claim 1, wherein the rigid arm member is bent at a bent region intermediate the first end region and a remaining end region.

7. The apparatus as claimed in claim 6, wherein the first end region has a respective first arm axis that is offset from a corresponding further arm axis of the remaining end region by about around 15° to 40°.

8. The apparatus as claimed in claim 7, wherein the first arm axis and the further arm axis are oriented at an angle of around 135° or 145°.

9. The apparatus as claimed in claim 1, wherein the mating threaded region comprises a threaded through bore in a protuberance of the arm member in the first end region.

10. The apparatus as claimed in claim 1, wherein the wedge element comprises a first sliding surface that faces a respective sliding surface provided by an outer surface of the first end region, and an abutment surface.

11. The apparatus as claimed in claim 10, wherein the abutment surface comprises a recessed channel region provided in an outer surface of the wedge element.

12. The apparatus as claimed in claim 11, wherein the recessed channel comprises a widened and a narrowed region, and the sliding surface of the wedge element comprises a protruding element, of a width that is narrower than the widened region and is wider than the narrowed region.

13. The apparatus as claimed in claim 11, wherein a primary axis associated with the first sliding surface is angled with respect to a primary axis associated with the channel region by about around 10° to 40°.

14. The apparatus as claimed in claim 1, wherein a remaining end region comprises at least one elongate through hole.

15. The apparatus as claimed in claim 1, wherein a remaining end region comprises at least one predominantly circular through hole for receiving an elongate bolt.

16. The apparatus as claimed in claim 1, wherein a remaining end region comprises an end that includes a flared-out impact receiving head.

17. The apparatus as claimed in claim 16, wherein the flared-out impact receiving head is oriented at an angle of between 5° and 20° with respect to a vertical axis.

18. The apparatus as claimed in claim 1, wherein a remaining end region comprises a plurality of discrete rigid spaced apart elongate members that extend in a substantially parallel relationship.

19. The apparatus as claimed in claim 18, wherein a radially outermost surface of each elongate member includes wrinkles or corrugations.

20. The apparatus as claimed in claim 1, wherein the locating member is removable and/or replaceable.

21. A method of urging a prosthetic femoral component into a desired position at a target location, comprising the steps of:
locating a rigid arm member with respect to a femoral component via a locating member at a first end region of the arm member;
turning a handle at a first end of a shaft member supported by the arm member thereby driving the shaft member and a wedge element carried at a remaining end of the shaft member, along a respective drive axis;
wherein:
as the shaft member is driven in a first drive direction, driving a narrowed portion of the wedge element between a region of the rigid arm member and a femoral neck portion of the femoral component thereby securing the arm member with respect to the femoral component; and
subsequently urging the prosthetic femoral head component into a desired position with respect to a femur.

22. The method of claim 21, further comprising locating a shaft of a prosthetic femoral component in a channel in a femur.

23. The method of claim 21, further comprising urging the prosthetic femoral component by hand or via a surgical mallet member by hitting a remaining end of the rigid arm member.

24. The method of claim 21, further comprising subsequent to the prosthetic femoral component being duly located at a desired position, turning the handle member to drive the wedge member and shaft member away from the femoral neck; and
urging the arm member away from the femoral component.

25. The method of claim 21, further comprising hitting the remaining end by impacting a flared-out end of the arm member with a surgical mallet.

26. The method of claim 21, further comprising locating the rigid arm member at a presentation position via manipulation of a plurality of elongate handle members at an end of the rigid arm member distal to the locating member.

27. Apparatus for implanting a prosthetic stem into a terminal end region of bone comprising:
an elongate body region; a stem securement region connected to the elongate body region comprising a slidable wedge element, for imparting a first securing force on the stem, and a screw element for selecting the position of said wedge element wholly or partly along the elongate body region; and
a stem coupling region comprising a protrusion element for coupling with the stem, wherein the protrusion element imparts a further securing force in response to the first securing force, thereby securing the stem to the apparatus.
